(19) United States
(12) Patent Application Publication
(10) Pub. No.: US 2006/0128614 A1
(43) Pub. Date: Jun. 15, 2006

(54) Antimicrobial peptides with reduced hemolysis and methods of their use

(76) Inventors: Jya-Wei Cheng, Coquitlam (CA); Shiou-Ru Tzeng, London (CA)

Correspondence Address: OYEN, WIGGS, GREEN & MUTALA LLP;480 - THE STATION, 601 WEST CORDOVA STREET, VANCOUVER, BC, V6B 1G1, CA

(21) Appl. No.: 11/000,970

(22) Filed: Dec. 2, 2004

Related U.S. Application Data
(63) Continuation-in-part of application No. 10/247,476, filed on Sep. 20, 2002, now abandoned.

(51) Int. Cl. A61K 38/12; A61K 38/08; C07K 5/12; C07K 7/06

(52) U.S. Cl. 514/9; 514/17; 530/317; 530/330

(57) ABSTRACT

The present invention provides novel cyclic and linear short peptides containing one of the following amino acid residue sequences: Xa₁-Naa-Xa₁-Xa₁-Naa-Xa₂ or Xa₁-Naa-Xa₂-Xa₁-Naa-Xa₁ wherein: Xa₁ represents lysine, arginine, or histidine; Naa represents an unnatural hydrophobic aromatic amino acid moiety selected from the group consisting of (naphtha-1-yl)alanine (1-Nal), (naphtha-2-yl)alanine (2-Nal), (benzothien-3-yl)alanine (Bal), diphenylalanine (Dip), (4,4′-biphen-yl)alanine (Bip), (anthracen-9-yl)alanine (Ath), and (2,5,7-tri-tert-butyl-indol-3-yl)alanine (Tht); and Xa₂ represents valine, leucine, or isoleucine. The novel peptides exhibit broad spectrum antimicrobial activity against Gram-positive and Gram-negative bacteria and fungi by effecting modification of the primary peptide structure. Further, the peptides exhibit improved serum compatibility and reduced hemolysis.

### RELATED APPLICATION

This application is a continuation-in-part of U.S. patent application Ser. No. 10/247,476 filed Sep. 20, 2002.

### TECHNICAL FIELD OF THE INVENTION

The invention relates to the field of antibiotic peptides. In particular, the invention is directed to cyclic and non-cyclic peptides having unnatural amino acid residues arranged in unique patterns of aromatic and cationic residues which exhibit very high antimicrobial efficacy and low hemolytic activity, as compared to other peptides having aromatic and cationic amino acid residues.

### BACKGROUND OF THE INVENTION

The emergence of bacterial strains that are resistant to conventional antibiotics has prompted a search for new therapeutic agents, including antimicrobial peptides of animal origin. Antimicrobial peptides have been recognized as playing an important role in the innate host defense mechanisms of most living organisms including those of plants, insects, amphibians and mammals, and are known to possess potent antibiotic activity against bacteria, fungi, and certain viruses.

As antimicrobial peptides are low molecular mass molecules of less than 5 kiloDaltons possessing broad-spectrum activity and constituting an important part of the host defense against microbial infections, they provide a starting point for designing low molecular mass antibiotic compounds.

Most antimicrobial peptides do not target specific molecular receptors of the pathogens, but rather interact and permeabilize microbial membranes. They are known to have a propensity to fold into amphipathic structures with clusters of hydrophobic and charge regions, a feature contributing to their membranolytic activity. The antimicrobial peptides readily partition into phospholipid bilayers with greater than 95% of the peptides binding to lipid to compromise membrane integrity. In bacteria, antimicrobial peptides are able to cause small, transient increases in conductance in planar lipid bilayers, thereby partially depolarizing the cytoplasmic membrane potential gradient. As bacteria increasingly develop antibiotic resistance, the potential for the development of antimicrobial peptides, as novel therapeutic agents which could overcome the problem of antibiotic resistance, is evident.

The protective function of antimicrobial peptides in innate host defense mechanisms has been demonstrated in Drosophila, where reduced expression of such peptides dramatically decreases survival rates after microbial challenge. In mammals, a similar function is suggested by defective bacterial killing in the lungs of cystic fibrosis patients and in small mice.

To date, several hundred antimicrobial peptides have been characterized. The naturally derived antimicrobial peptides are, generally, between 12 and 50 amino acids in length and are folded into several structural groups including α-helices, β-sheets, extended peptides and looped peptides. Although these peptides show a marked degree of variability, they possess two common and functionally important requirements: a net cationicity that facilitates interaction with negatively charged microbial surfaces, and the ability to assume amphipathic structures which permit incorporation into microbial membranes.

The antimicrobial peptides found in mammals may be classified into the cysteine-rich defensins (α- and β-defensin) and various groups within the cathelicidin family. Based on the amino acid composition and structure, the cathelicidin family may be classified into three groups. The first group includes the amphipathic α-helical peptides such as LL-37, CRAMP, SMAP-29, PMAP-37, BMAP-27, and BMAP-28. The second group contains the Arg/Pro-rich or Trp-rich peptides including BacS, Bac7, PR-39, and indolicidin. The third group includes Cys-containing peptides such as protegrins. Cathelicidin families contain a highly-conserved signal sequence and proregion known as the cathelin domain and a variable antibacterial sequence in the C-terminal domain. Many cathelicidins contain a characteristic elastase cleavage site between the anionic cathelin domain and the cationic C-terminal peptide domain. Proteolytic processing at this site has been observed in bovine and porcine neutrophils and is required for microbicidal activity.

Tryptophan has a high potency to insert into membranes and to cause Trp-rich peptides to partition into membranes which affects lipid polymorphism. These important features of Trp residues in antimicrobial and hemolytic activity have allowed the structure and mechanism of action of several Trp-rich cationic antimicrobial peptides to be determined. A well-studied example of Trp-rich antimicrobial peptides is indolicidin. Indolicidin, with the amino acid sequence Ac-ILPWKWPWWPWRR-NH₂, is a short Trp-rich peptide isolated from the cytoplasmic granules of bovine neutrophils. Indolicidin has an extended, boat-shaped structure and wide-spectrum antimicrobial activity against Gram-positive and Gram-negative bacteria, protozoa, fungi, and the enveloped virus HIV-1. A similar mechanism of action has been proposed for other Trp-rich peptides such as tritrpticin, puroA, lactoferricin, and PW2.

### SUMMARY OF THE INVENTION

The present invention is directed to the design of antibiotic peptides having broad spectrum antimicrobial activity against clinically important Gram-positive and Gram-negative bacteria, and fungi, by synthetic modification of the primary structure of the peptides. The invention is further directed to the design of cyclic and short peptides with improved serum compatibility and reduced hemolytic activity. Furthermore, the present invention exhibit broad-spectrum antimicrobial activity against Gram-positive and Gram-negative bacteria and multi-drug-resistant pathogens and low hemolytic activity.

In one of its aspects, the invention comprises antimicrobial peptides generally composed of unnatural amino acid residues and containing at least one of the following amino acid sequences:
Xa₁-Naa-Xa₁-Xa₁-Naa-Xa₂
   or
Xa₁-Naa-Xa₂-Xa₁-Naa-Xa₁
   wherein:
Xa₁ represents a hydrophilic basic amino acid moiety selected from the group consisting of lysine, arginine, and histidine,
Naa represents an unnatural hydrophobic aromatic amino acid moiety selected from the group consisting of (naphtha-1-yl)alanine (1-Nal), (naphtha-2-yl)alanine (2-Nal), (benzothien-3-yl)alanine (Bal), diphenylalanine (Dip), (4,4′-biphen-yl)alanine (Bip), (anthracen-9-yl)alanine (Ath), and (2,5,7-tri-tert-butyl-indol-3-yl)alanine (Tht),
Xa₂ represents a hydrophobic amino acid moiety selected from the group consisting of valine, leucine, and isoleucine.

In another aspect, the invention comprises an isolated and purified linear or cyclic peptide chosen from the group of peptides of claim 1, which is selected from the group consisting of

| | |
|---|---|
| K(2-Nal)RR(2-Nal)I | (SEQ ID NO:1) |
| K(2-Nal)RR(2-Nal)V | (SEQ ID NO:2) |
| K(2-Nal)IK(2-Nal)R | (SEQ ID NO:3) |
| K(2-Nal)RR(2-Nal)VR(2-Nal)I | (SEQ ID NO:4) |
| I(2-Nal)RV(2-Nal)RR(2-Nal)K | (SEQ ID NO:5) |
| K(1-Nal)RR(1-Nal)VR(1-Nal)I | (SEQ ID NO:6) |
| K(Bip)RR(Bip)VR(Bip)I | (SEQ ID NO:7) |

In others of its aspects, the isolated and purified peptide of claim 1 may be amidated at the C-terminal amino acid, acetylated at the N-terminal amino acid, or esterified at the C-terminal amino acid, or at least one amino acid may be altered to a corresponding D-amino acid.

In another of its aspects, the invention comprises a composition comprising an isolated and purified peptide according to claim 1 in a mixture with a carrier or excipient.

In still further of its aspects, the invention comprises a method to inactivate an endotoxin of a Gram-negative bacteria comprising the step of administering an isolated and purified antimicrobial peptide according to claim 1 to a subject in need thereof, a method to treat a microbial or viral infection in a subject comprising the step of administering an isolated and purified antimicrobial peptide according to claim 1 to a subject in need thereof, and a method to inhibit the growth of a microbe comprising the step of administering an isolated and purified antimicrobial peptide according to claim 1 to a subject in need thereof.

In yet another of its aspects, the invention provides a pharmaceutical composition which comprises a peptide of claim 1 and a pharmaceutical carrier.

In still a further aspect, the invention comprises a peptide which is the retro-oriented amino acid sequence of a peptide of claim 1.

In other aspects, the invention comprises a peptide of claim 1 which is of the formula Lys-(2-Nal)-Arg-Arg-(2-Nal)-Val-Arg-(2-Nal)-Ile, and a pharmaceutical composition which comprises a peptide of the formula Lys-(2-Nal)-Arg-Arg-(2-Nal)-Val-Arg-(2-Nal)-Ile.

The invention provides several advantages. First, the peptides of the invention are less than 10 amino acid residues and extremely compact so that it is effective to span the cell membrane with relatively few amino acids. Secondly, the best peptides from the invention exhibit strong broad-spectrum activity against antibiotic resistant bacteria, combined with activity against the medically important fungi. In addition, these peptides possess anti-endotoxin activity and work synergistically with traditional antibiotics. Most importantly, the invention offers improved serum compatibility and exhibits extremely low hemolysis against human red blood cells as compared with the naturally-occurring protegrins and indolicidin analogs.

### BRIEF DESCRIPTION OF THE FIGURES

The invention will be described by reference to the preferred embodiment and the experimental data presented in the figures, in which:

FIG. 1(A) is the survival curve for Bacillus subtilis ATCC6633 treated with Pac-625 (solid circle) and Pac-626 (open circle);

FIG. 1(B) is the survival curve for Staphylococcus aureus ATCC9144 treated with Pac-625 (solid circle) and Pac-626 (open circle);

FIG. 1(C) is the survival curve for Escherichia coli ATCC25922 treated with Pac-625 (solid 5 circle) and Pac-626 (open circle);

FIG. 1(D) is the survival curve for Pseudomonas aeruginosa ATCC29213 treated with Pac-625 (solid circle) and Pac-626 (open circle);

FIG. 2 shows Pac-625 induced inner membrane permeabilization assessed by NPN uptake (0 μg/ml (solid square); 1 μg/ml (open circle) 2 μg/ml (open square); and 3 μg/ml (open triangle);

FIG. 3(A) shows fluorescence emission spectra for 1.5 μM Pac-625 bound to 25 mM SDS (solid circle) and in buffer (open circle); 15

FIG. 3(B) shows Stern-Volmer plots for 1.5 μM Pac-625 bound to 25 mM SDS (solid circle) and in buffer (open circle);

FIG. 4 shows circular dichroism spectra for 100 μM Pac-625 bound to 10 mM SDS, 10 mM DPC, 1 mM LPC12, 50% TFE, and in buffer at 25° C.;

FIG. 5 shows the hemolytic activity against human red blood cells of melittin (solid circle), Pac-625 (open circle) and Pac-626 solid triangle).

### DETAILED DESCRIPTION OF BEST MODE AND PREFERRED EMBODIMENT OF THE

25 INVENTION

As with the Trp-rich peptides described in U.S. Pat. No. 6,303,575; U.S. Pat. No. 6,180,604; U.S. Pat. No. 5,821,224; U.S. Pat. No. 5,547,939; U.S. Pat. No. 5,534,939; U.S. Pat. No. 5,459,325, U.S. Pat. No. 5,324,716, and WO93/14115, all antimicrobial peptides having natural amino acid residues undergo rapid proteolysis in vivo. Indolicidin analogues have the general amino acid sequence Ac-1-L-P-W-K-W-P-W-W-P-W-X, where X represents 1 or 2 selected amino acids. These previously described Trp-rich peptides are distinguishable from those of the present invention in that the design of antimicrobial peptides of the present invention is directed to introduce unnatural amino acid residues at specific sequence positions to cyclic or linear peptides having less than 10 amino acids and having broad-spectrum microbicidal activity, improved selectivity and low hemolysis.

It was found that the antibacterial activity of Trp-rich peptides is not dependent on the presence of Trp residues per se, but rather that the size and shape of the aromatic moiety of the amino acid (Haug et al., Journal of Peptide Science, vol. 7, pp. 425-432, 2001). The introduction of large unnatural aromatic amino acids as replacements for Trp of lactoferricin derivatives gave an increase in the antibacterial activity (Haug et al., Journal of Peptide Science, vol. 7, pp. 425-432, 2001). Furthermore, it has been shown that the hydrophobicity of the antimicrobial peptides is closely related to their hemolytic activity. For indolicidin, the replacement of all five Trp residues with Phe results in reducing the hemolytic activity, whereas the antibacterial activity is retained (Subbalakshmi et al., FEBS Letters, vol. 395, pp. 48-52, 1996). Thus, introducing unnatural aromatic amino acids that have larger volumes than Trp into antimicrobial peptides can increase their antibacterial activity and subsequently reduce their hemolytic activity.

As with the cyclic antimicrobial peptides described in WO99/21879A1 and WO01/68675A3, cyclization of linear antimicrobial peptides may have several advantages with regard to selectivity and stability. Firstly, unfolded peptides may form aggregates due to hydrophobic interactions, leading to non-specific adsorption to normal mammalian cells and to low solubility. Placing constraints on their unfolded conformations, thereby restricting exposure of hydrophobic stretches of amino acids, can limit these hydrophobic interactions. Furthermore, these constraints may enhance the role of electrostatic interactions in initial binding with the negatively-charged target membrane, thus substantially increasing selectivity for bacteria over mammalian cells. Secondly, to be bound and cleaved by protease, a peptide must present the cleavage site in an extended, unfolded structure. Thus, cyclization of short peptides may limit their accessibility to protease activity due to their rigid and constrained structure. Since cyclization seems to affect activity only in relation to Gram-negative bacteria, further studies may assist in the design of bacteria-specific lytic peptides.

In most of the membrane-lytic peptides, the distribution and amount of net charge correlate with their biological function. For example, most peptides with a low net negative or low net positive charge distributed along their helix backbone are lytic to mammalian cells or to both mammalian and bacterial cells. On the other hand, low-hemolytic antimicrobial peptides tend to contain high net positive charge contributed by a large number of basic amino acids that are distributed along the hydrophilic face of the amphipathic α-helix.

The NMR structures of indolicidin have been determined in both DPC and SDS micelles. In both micelles, indolicidin adopts an extended structure with the positively charged residues Arg and Lys localized at the ends of the structure and the Trp residues forming a hydrophobic core. The NMR structure of another Trp-rich antimicrobial peptide, tritrpticin, with the amino acid sequence VRRFPWWWPFLRR, shows an amphipathic turn-turn structure with the Trp residues clustered together and inserted in the hydrophobic core of the SDS micelle. Residues 4 to 9 of the lactoferricin peptide (LfcinB₄₋₉) with the sequence RRWQWR-NH₂, has been shown to display antimicrobial activity which compares favorably to LfcinB. LfcinB₄₋₉ has been found to be form a stable amphipathic structure in SDS micelles, with the Trp side-chains located deeper within the micelle than the Arg and Gln residues.

In an effort by the present inventors to develop more effective and low molecular mass antimicrobial peptides, a linear peptide library with an amino acid length ranging from three to nine residues using tryptophan as a template at various positions was designed and synthesized. Of the newly designed peptides, the nine amino acid residue peptide, Ac-KWRRWVRW₁-NH₂, designated as Pac-525, demonstrates improved activity against both Gram-positive and Gram-negative bacteria, as well as reduced hemolytic activity. The solution structures of Pac-525 bound to membrane-mimetic SDS and DPC micelles have been determined by two-dimensional NMR methods. The SDS micelle-bound structure of Pac-525 adopts a 3₁₀ α-helix at residues Trp2, Arg3 and Arg4. The positively charged residues were clustered together to form a hydrophilic patch. The three hydrophobic residues including Trp2, Val6, and Ile9 form a hydrophobic core. The surface electrostatic potential map indicates the three tryptophan indole rings are packed against the peptide backbone to form an amphipathic structure. A cationic amphipathic structure would be best suited for maximizing both electrostatic and hydrophobic interactions with a membrane.

While viewing the amino acid sequence and the solution structure of Pac-525, it was observed that the reversed sequence of Pac-525, Ac-IWRVWRRWK-NH₂, designated as Pac-525ᵣₑᵥ, contains the same amino acids and also possess similar antimicrobial activity as Pac-525. In addition, the hemolytic activity of Pac-525ᵣₑᵥ is very similar to that of Pac-525. As determined by NMR, both the Pac-525 and Pac-525ᵣₑᵥ adopt similar α-helical structures. Furthermore, both exhibit amphipathic structures with basic amino acids on one side and hydrophobic amino acids on the other side. These structures offer an explanation for the high potency of the antimicrobial activities and the low hemolytic activities of Pac-525 and Pac-525ᵣₑᵥ.

The present invention provides a further modification of Pac-525 to increase its antibacterial activity and reduce its hemolytic activity by introducing unnatural amino acid residues, and to increase its stability against proteases by means of cyclization and by replacing the naturally-occurring L-form amino acids with D-form amino acids.

### Example 1

### Design, Synthesis, Purification and Characterization of Peptides

Linear peptides were synthesized by solid-phase peptide synthesis using the standard Fmoc (N-(9-fluoroenyl)methoxycarbonyl) protocol manually on PAL resin (5-(4-Fmoc-aminomethyl-3,5-dimethoxyphenoxy-valeric acid-MBHA). The coupling reaction was permitted to occur for about 1 to 1.5 hours and checked by the ninhydrin test. Removal of the N-terminal Fmoc protecting group was accomplished by gentle stirring with 20% piperidine in dimethylformamide for 2 hours at room temperature. The acetylation of the peptides was achieved by adding 10-fold acetic anhydride and 20-fold DIPEA in dimethylformamide and stirring for 2 hours at room temperature. The cleavage of peptides from the resin was carried out by mixing with 95% TFA cleavage mixture for 1 to 1.5 hours. The cyclic peptides of the invention may be prepared using any art-known technique for the preparation of cyclic peptides. For example, the linear peptides prepared by solid-phase peptide synthesis can be cyclized using standard chemistry. Preferably, the chemistry used to cyclize the linear peptide will be sufficiently mild so as to avoid substantially degrading the peptide. Suitable procedures for synthesizing the peptides described herein as well as suitable chemistry for cyclizing the peptides are well known in the art and can be found in the references of Dong et al., 1995, J. Am. Chem. Soc. 117, pp. 2726-2731, Ishida et al., 1995, J. Org. Chem., 60, pp. 5374-5375, Toniolo, 1990, Int. J. Peptide Protein Res., 35, pp. 287-300 and WO99/21879A1. The crude peptides were then purified by reverse phase high pressure liquid chromatography (RP-HPLC) using a Vydac C₁₈ reversed-phase column. The mobile phase for elution was a mixture of acetonitrile and deionized H₂O mixed in different ratios using the built-in gradient. The wavelength for detection was set at 225 nm and 280 nm, and the flow rate for elution was 4 ml/min. The major peptide products were characterized by fast atom bombard mass spectrophotometry to determine the molecular weight of each peptide. The purity of each peptide was analyzed by analytical RP-HPLC. The identity of the peptides was checked by electrospray mass spectroscopy.

The peptides of the invention can be described by one of the following formulae:
Xa₁-Naa-Xa₁-Xa₁-Naa-Xa₂
   or
Xa₁-Naa-Xa₂-Xa₁-Naa-Xa₁
   wherein:
Xa₁ represents a hydrophilic basic amino acid moiety selected from the group consisting of lysine, arginine, and histidine,
Naa represents an unnatural hydrophobic aromatic amino acid moiety selected from the group consisting of (naphtha-1-yl)alanine (1-Nal), (naphtha-2-yl)alanine (2-Nal), (benzothien-3-yl)alanine (Bal), diphenylalanine (Dip), (4,4′-biphen-yl)alanine (Bip), (anthracen-9-yl)alanine (Ath), and (2,5,7-tri-tert-butyl-indol-3-yl)alanine (Tht),
Xa₂ represents a hydrophobic amino acid moiety selected from the group consisting of valine, leucine, and isoleucine.

The topology of the peptides of the invention may be either cyclic or linear.

Peptide analogues of the invention and their assigned names are listed in Table 1. All of the amino acids except the unnatural amino acids are denoted by the one-letter amino acid code.

TABLE 1

Peptide analogues of the invention

Sequence

| | |
|---|---|
| Identification | Peptide |

| | | |
|---|---|---|
| No. | name | Amino acid sequence |

| | | |
|---|---|---|
| 1 | Pac-401 C | K (2-Nal) R R (2-Nal) I |
| 1 | Pac-401 L | K (2-Nal) R R (2-Nal) I |
| 2 | Pac-402 C | K (2-Nal) R R (2-Nal) V |
| 2 | Pac-402 L | K (2-Nal) R R (2-Nal) V |
| 3 | Pac-403 C | K (2-Nal) I K (2-Nal) R |
| 3 | Pac-403 L | K (2-Nal) I K (2-Nal) R |
| 4 | Pac-625 C | K (2-Nal) R R (2-Nal) V R (2-Nal) I |
| 4 | Pac-625 L | K (2-Nal) R R (2-Nal) V R (2-Nal) I |
| 5 | Pac-626 C | I (2-Nal) R V (2-Nal) R R (2-Nal) K |
| 5 | Pac-626 L | I (2-Nal) R V (2-Nal) R R (2-Nal) K |
| 6 | Pac-627 C | K (1-Nal) R R (1-Nal) V R (1-Nal) I |
| 6 | Pac-627 L | K (1-Nal) R R (1-Nal) V R (1-Nal) I |
| 7 | Pac-628 C | K (Bip) R R (Bip) V R (Bip) I |
| 7 | Pac-628 L | K (Bip) R R (Bip) V R (Bip) I |

Note:
C and L denote cyclic or linear topology.

The peptides of the present invention may be acetylated at the N-terminus, amidated or esterified at the C-terminus, or synthesized as their D-amino acid analogs. It is well known in the art of peptide synthesis to acetylate the N-terminus of a peptide by reacting the final peptide with acetic anhydride before cleavage from the resin or to amidate the C-terminus of a peptide using an appropriate resin such as methylbenzhydrylamine resin using Boc (butoxycarbonyl). Further, it is well known in the art to esterify the C-terminal amino acid of a peptide. It is also well known in the art that peptides containing the D-amino acids may be synthesized by replacing the L-amino acids with D-amino acids during peptide synthesis. All resins and amino acids are available for purchase from Novabiochem™ or Synpep™.

### Example 2

### Determination of Peptide Activity In Vitro

The in vitro antimicrobial activities of antimicrobial agents were tested using standard NCCLS bacterial and fungi inhibition assays or minimum inhibition concentration (MIC) tests. The MIC value is the lowest concentration of peptide at which the visible growth of test organisms was inhibited and reduced. The test organisms used in the MIC assays are listed in Table 2.

TABLE 2
Test strains used for MIC determination

| Organism | Source |
|---|---|
| Bacillus subtilis | ATCC 6633 |
| Staphylococcus aureus | ATCC 9144 |
| Staphylococcus epidermidis | ATCC 12228 |
| Staphylococcus aureus | ATCC 29737 |
| Bacillus pumilus | ATCC 14884 |
| Bacillus cereus | ATCC 11778 |
| Pseudomonas aeruginosa | ATCC 29213 |
| Staphylococcus aureus | ATCC 29213 |
| Escherichia coli | ATCC 25922 |
| Candida albicans | ATCC 10231 |

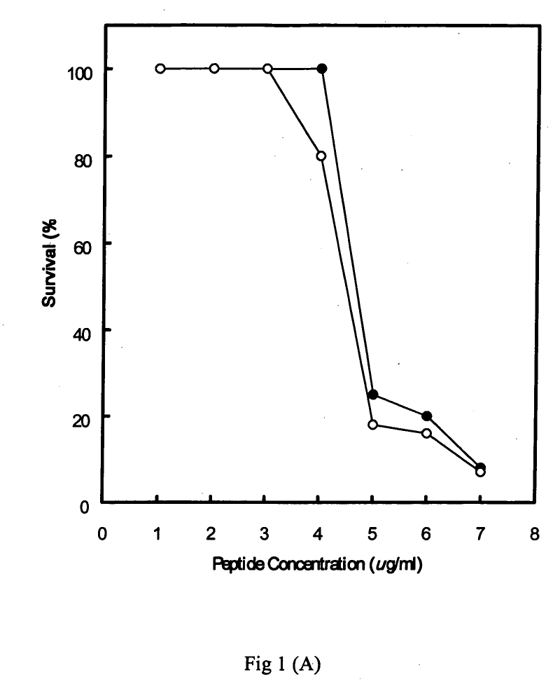

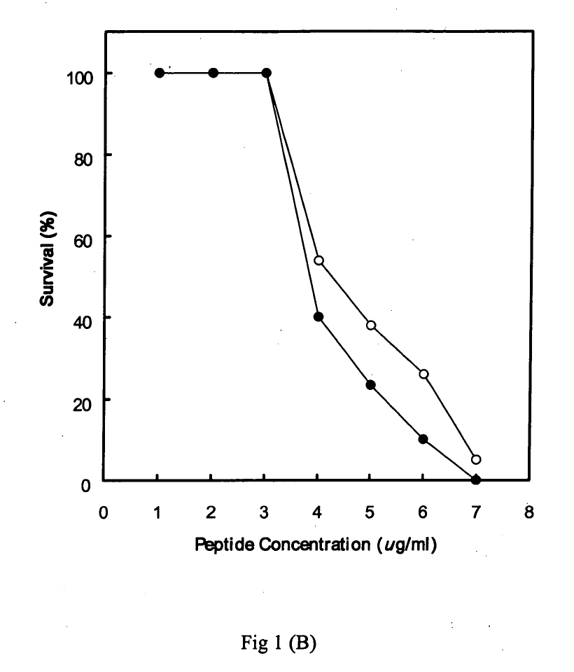

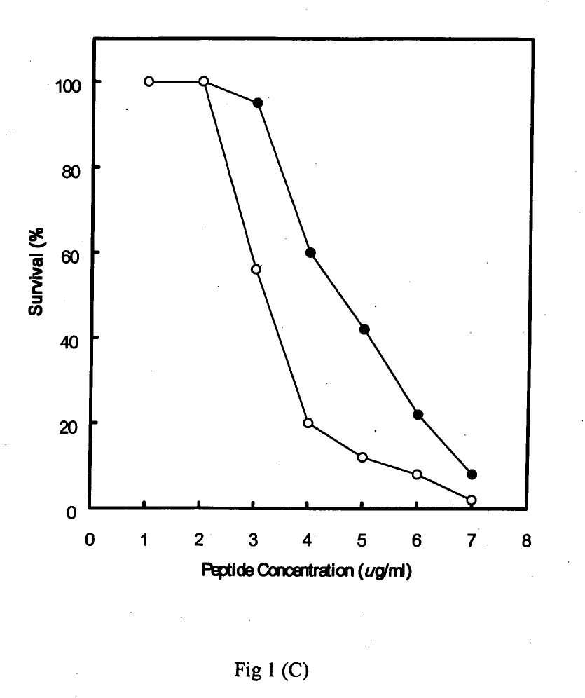

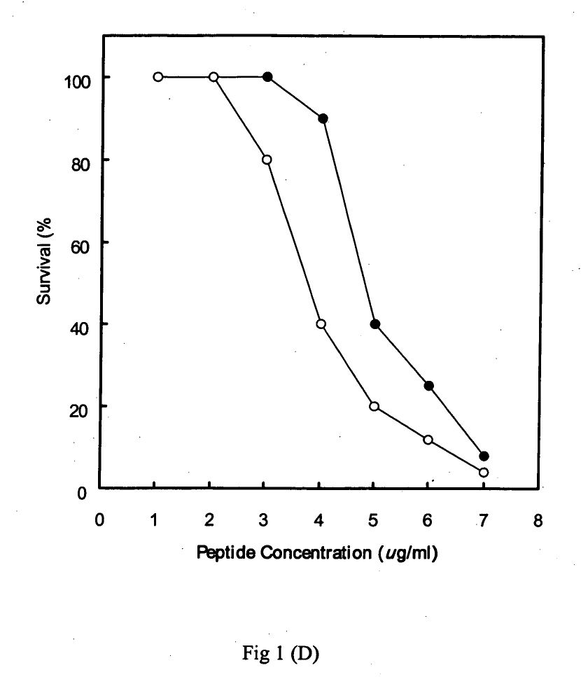

Overnight cultures of the test organisms were diluted to produce an inoculum containing approximately 105 colonies in Meuller-Hinton broth (MHB). From the peptide stock solution, serial dilutions of the peptides into 501 volumes were prepared and all wells of a 96-well microtiter plate were subsequently inoculated with the diluted culture of the test organisms. After 18 hours of incubation at 37°, the results were assayed for turbidity as an indicator of cell growth. MIC values for the peptides are shown in Table 3 and Table 4 and FIGS. 1(A), 1(B), 1 (C) and 1 (D) show survival rates for Pac-625 and Pac-626.

TABLE 3
MIC (μg/ml) value for synthetic peptides
against E. coli and S. aureus

| Peptide name | E. coli | S. aureus |
|---|---|---|
| Pac-401 C | >64 | >64 |
| Pac-401 L | 16 | 8 |
| Pac-402 C | >64 | >64 |
| Pac-402-L | 24 | 16 |
| Pac-403-C | >64 | >64 |
| Pac-403-L | 24 | 16 |
| Pac-625 C | 64 | 64 |
| Pac-625 L | 2 | 4 |
| Pac-626 C | 64 | 64 |
| Pac-626 L | 4 | 4 |
| Pac-627 C | 64 | 64 |
| Pac-627 L | 4 | 4 |
| Pac-628 C | 64 | 64 |
| Pac-628 L | 2 | 4 |

Note:
C and L indicate cyclic or linear topology

Table 3 depicts the mean MIC values based on three separate MIC tests. Pac-625, Pac-626, Pac-627, and Pac-628 showed the greatest antimicrobial activity against Gram-positive and 5 Gram-negative bacteria, and showed greater microbicidal activities than did Pac-401, Pac-402, or Pac-403. Table 4 shows the MIC (μg/ml) values for Pac-625 and Pac-626 against various bacteria and fungi strains.

TABLE 4
MIC (μg/ml) values for Pac-625 and Pac-626 against
various bacteria and fungi strains

| | Non Phosphate Buffer | | 1X Phosphate Buffer | |
|---|---|---|---|---|
| Organism | Pac-625 L | Pac-626 L | Pac-625 L | Pac-626 L |
| B. subtilis | 4 | 4 | 4 | 2 |
| S. epidermidis | 2 | 2 | 2 | 4 |
| S. aureus | 4 | 4 | 8 | 4 |
| B. pumilus | 4 | 2 | 8 | 4 |
| B. cereus | 2 | 4 | 8 | 4 |
| P. aeruginosa | 2 | 4 | 4 | 8 |
| E. coli | 2 | 4 | 8 | 8 |
| Candida albicans | 16 | 32 | 16 | 32 |

### Example 3

### Membrane Permeabilization Assays

The outer membrane permeabilization activity of the peptide variants was determined by the 1-N-phenylnaphthylamine (NPN) uptake assay, using intact cells of E. coli. NPN exhibits weak fluorescence in an aqueous environment but exhibits strong fluorescence in a hydrophobic environment. Since NPN is hydrophobic, it provides a direct measurement of the degree of outer membrane permeability. E. coli take up little or no NPN in a general condition. In the presence of permeabilizer compounds (EDTA, polymyxin B, Neomycin, or antimicrobial peptides), NPN partitioned into the bacterial outer membrane results in an increase in fluorescence. Fluorescence will vary with the concentration of peptide.

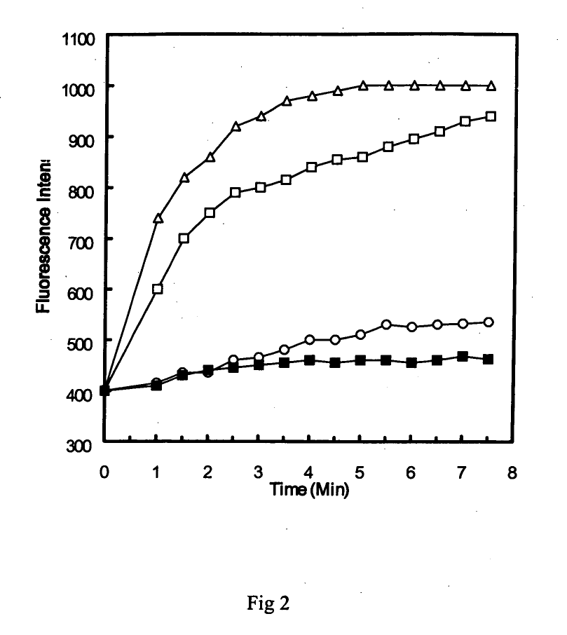

One ml of overnight culture was used to inoculate 50 ml of media and incubated at 37° C. with shaking. The culture was permitted to grow to an OD600 of 0.4 to 0.6, cells were spun down at 3500 rpm for 10 minutes, washed, and re-suspended in buffer to an OD600 of 0.5. The OD600 was recorded, 1 ml of cells (OD600=0.5) was added to the cuvette and measured after 2-5 seconds. 20 μl NPN 0.5 mM, shaken to mix, was added and measured after 2-5 seconds. 10 ul antibiotic 100× desired final concentration was added, shaken to mix, and measured until the maximal value was reached within 1 to 5 minutes. The concentration of peptide leading to 50% of the maximum increase in NPN uptake was recorded as the P₅₀. All of the peptides were capable of interacting with membrane, as demonstrated by the NPN uptake assay results shown in Table 5. FIG. 2 shows the membrane permeabilization of Pac-625 at various NPN concentrations.

TABLE 5
Ability to permeablize and promote NPN uptake
across outer membrane of E. coli

| Peptide name | P₅₀ (μg/ml) |
|---|---|
| Pac 401 L | 8 |
| Pac 402 L | 16 |
| Pac 403 L | 8 |
| Pac 625 L | 2 |
| Pac 626 L | 2 |
| Pac 628 L | 4 |

### Example 4

### Characterization of the Environment of the Naphthylalanine Residues

Because of the sensitivity of tryptophan to the polarity of its environment, it has been used for polarity and binding studies. In a first set of experiments to determine the environment of the peptides, the fluorescence emission spectrum of tryptophan was monitored in PBS at pH 7.4 and in the presence of vesicles composed of either PE/PG (7:3, w/w), a phospholipid composition typical of E. coli, or PC/cholesterol (10:1, w/w), a phospholipid composition used for mimicking the outer leaflet of human erythrocytes. Fluorescence spectra and emission intensities were measured with a Perkin-Elmer 55S fluorescence spectrophotometer equipped with a circulating water bath to maintain the cuvette holding chamber at 25° C. In these fluorometric studies, the lipid/peptide molar ratio was maintained at 1000:1 so that the spectral contributions of free peptide would be negligible. The results of this study are summarized in Table 6. In buffer, the 2-Nal and 1-Nal residues of Pac 625, Pac 626 and Pac 627 exhibited a maximum of fluorescence emission at around 349+2 nm, which reflects a hydrophilic environment for NaI. When PE/PG vesicles were added to the aqueous solutions containing the peptides, blue shifts in the emission maxima were observed for all peptides. Under our experimental conditions, the peptides exhibited a maximum of fluorescence emission at around 334+2 nm. The change in the spectrum of the Nal residue reflects its relocation to a more hydrophobic environment. In the presence of PC/cholesterol vesicles, no blue shift was observed for the peptides, indicating that these peptides do not bind PC/cholesterol vesicles or alternatively, bind them very weakly.

TABLE 6
Nal emission maximum of the peptides in buffer solution or in the
presence of different phospholipid composition

| Peptide name | PBS | PE/PG | PC/cholesterol | SDS |
|---|---|---|---|---|
| Pac 625 L | 350 ± 2 | 334 ± 2 | 338 ± 2 | 346 ± 2 |
| Pac 626 L | 351 ± 2 | 334 ± 2 | 339 ± 2 | 347 ± 2 |
| Pac 627 L | 350 ± 2 | 334 ± 2 | 339 ± 2 | 346 ± 2 |

In a second set of experiments, fluorescence emission spectra were recorded on an LS55 spectrofluorimeter (Perkin-Elmer). Measurements were performed between 300 and 450 nm within 0.5 nm increments using a 4×10-mm quartz cell at 25° C. The excitation wavelength was set to 295 nm with both the excitation and emission slit widths set to 5 nm. The concentration of peptide samples was 1.5 μM in 20 mM sodium phosphate buffer (pH 4.5) in the presence or absence of 25 mM SDS. Spectra were base-line-corrected by subtracting blank spectra of the corresponding solutions without peptide. For fluorescence quenching experiments, acrylamide was used as the quencher. Acrylamide was added so as to ensure final concentrations were between 2 and 116.5 mM. The quenched samples were excited at 295 nm, and the emission was monitored at the peak maximum determined from the wavelength scan in the absence of quencher. The effect of acrylamide on the fluorescence of the peptide was analyzed using the modified Stem-Volmer equation:
F₀/F=1+Kₛᵥ[Q]
where Kₛᵥ is the Stem-Volmer quenching constant and [Q] is the quencher concentration.

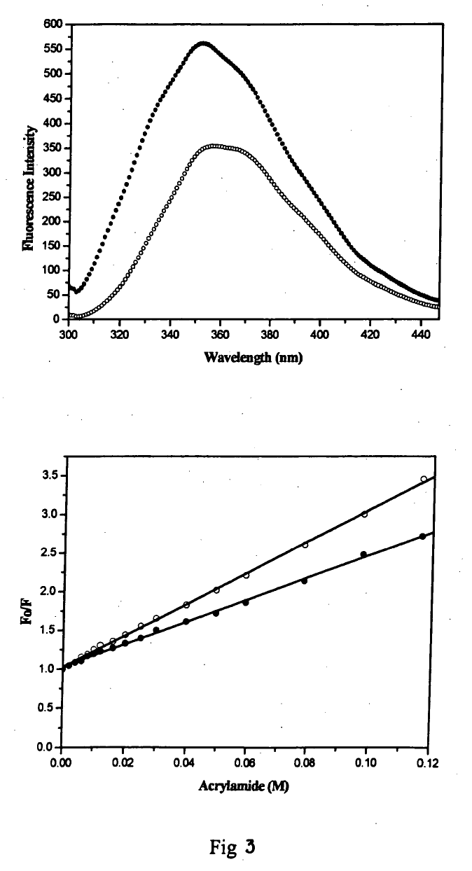

Owing to the sensitivity of Nal to the polarity of its environment, fluorescence spectroscopy has been used for polarity and micelle binding studies of aromatic-rich antimicrobial peptides. In the phosphate buffer, the fluorescence spectrum of Pac-625 exhibits an emission maximum at 356.5 nm (FIG. 3A). Whereas by adding SDS, its fluorescence spectrum displayed a 5.0 nm blue shift with a concomitant increase in intensity (FIG. 3A). This blue shift indicates that the Nal residues of Pac-625 were positioned into a more hydrophobic environment. The increase of fluorescence intensity suggests that the tryptophan residues were more sterically confined. In general, the fluorescence intensity increases as the polarity of the environment decreases.

To determine the extent to which the Nal residues are sequestered in the hydrophobic core of the micelle, a fluorescence quenching experiment was performed using acrylamide. The effective Stem-Volmer constants (K_{SV} (eff)) for Pac-625 were calculated to be 20.06±0.023 and 14.97±0.023 M⁻¹ in the absence and presence of SDS micelles (FIG. 3B). These values confirm that when the peptide is free in solution, it is more accessible to the quencher than in the presence of the micelles. The protection of the Nal residues by the micelles from acrylamide suggests that they are partially buried in the hydrophobic core of the micelles.

### Example 5

### Secondary Structure of the Peptides Determined by CD Spectroscopy

In a first experiment, CD spectra were recorded on an AVIV 62DS spectropolarimeter after calibration with d-10-camphorsulfonic acid. All measurements were carried out using an 1-mm path-length cuvette at a peptide concentration of 30 μM in 10 mM sodium phosphate buffer of pH 7.2. Far-UV spectra were collected in the range of 190-260 nm using a 0.5-nm step size and one second averaging time. In the absence of phospholipid, Pac-401, Pac-403, Pac-625 and Pac-626 are characterized by unordered structure, as indicated in Table 7.

TABLE 7
Peptide conformation in phosphate buffer conformation in SDS

| Peptide name | phosphate buffer | SDS |
|---|---|---|
| Pac 401 C | unordered | unordered |
| Pac 401 L | unordered | slightly structured |
| Pac 403 C | unordered | unordered |
| Pac 403 L | unordered | slightly structured |
| Pac 625 C | unordered | unordered |
| Pac 625 L | unordered | slightly structured |
| Pac 626 C | unordered | unordered |
| Pac 626 L | unordered | slightly structured |

In addition, CD spectra for Pac-625 were carried out in water or aqueous SDS, DPC, LPCl2 (Lysophosphatidylcholine), and TFE at pH 4.5 and 25° C. using a 1 mm path length cuvette. Three scans were averaged for each spectrum with a 0.2 nm stepsize. Peptide concentrations were 100 μM obtained by a quantitative serial dilution of a 10 mM stock solution. The mean residue ellipticity at 222 nm, in deg·cm²/dmol, was calculated from [θ]₂₂₂=θ/lcn, where θ is the ellipticity observed at 222 nm, 1 is the path length of the cell, c is the concentration of sample, and n is the number of peptide bonds in the sequence. The fraction of helix was obtained from the relationship f=[θ]_{obs}/[θ]ₘₐₓ. [θ]_{obs} is the mean residue ellipticity observed at 222 nm, and [θ]ₘₐₓ=((n−4)/n)[θ]_{∞}=the maximal mean residue ellipticity value for chain length n, where [θ]_{∞}=−40,000 deg·cm²/dmol.

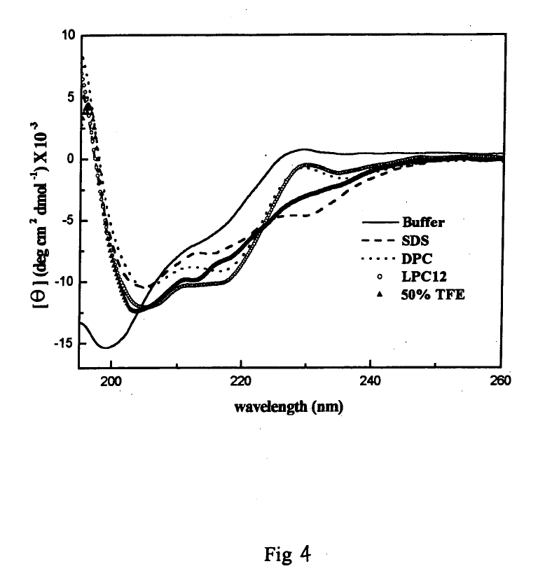

The CD spectra of Pac-625 recorded with and without SDS, DPC, LPC12 (Lysophosphatidylcholine), and TFE are shown in FIG. 4. In aqueous solution, the spectrum presents a negative band at 200 nm indicating a typical random coil conformation. The addition of micelles of SDS, DPC, and LPC12 as well as TFE led to a dramatic structure change resulting in a more ordered structure. This is supported by the fact that the negative band at 200 nm became less negative and an increasing negative band at 220 nm was observed in the presence of the micelles and TFE.

Table 8 shows the structure-activity relationship of Pac-625 and its derivatives. It was found that the decrease in positively charged amino acids (Pac-621 and Pac-629) abates their antimicrobial activities. Alternatively, the replacement of the 2-Nal residues of Pac-625 causes minimum or no effect on their antimicrobial activities (Pac-627 and Pac-628). Bacterial membranes are rich in acidic phospholipids. Therefore, the net positive charge of the antimicrobial peptides facilitates their perturbing activity toward bacterial membrane. In contrast, the outer membrane of human erythrocytes is composed predominantly of zwitterionic phosphatidylcholine and sphingomyelin phospholipids. In the case of Pac-625, four out of the nine residues are positively charged amino acids. This may explain the broad antimicrobial activity and low hemolytic activity of Pac-625.

TABLE 8

MIC (μg/ml) value for Pac-625 and its derivatives against
E. coli and S. aureus

| | | | |
|---|---|---|---|
| Name | Sequence | E. coli | S. aureus |

| | | | |
|---|---|---|---|
| Pac-625 | Ac-K (2-Nal) R R (2-Nal)VR (2-Nal) I-NH₂ | 2 | 4 |
| Pac-626 | Ac-I(2-Nal) RV(2-Nal) RR(2-Nal) K-NH₂ | 4 | 4 |
| Pac-621 | Ac-K(2-Nal) IK(2-Nal) IK(2-Nal) I-NH₂ | 8 | 16 |
| Pac-629 | Ac-K(2-Nal) IR(2-Nal) VR(2-Nal) I-NH₂ | 8 | 4 |
| Pac-627 | Ac-K(1-Nal) RR(1-Nal) VR(1-Nal) I-NH₂ | 4 | 4 |
| Pac-628 | Ac-K(Bip)RR(Bip)VR(Bip)I-NH₂ | 2 | 4 |

### Example 8

### Erythrocyte Lysis

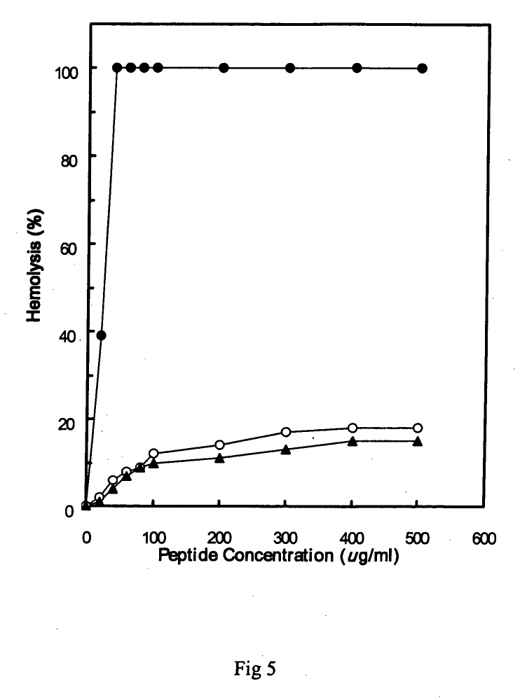

Pac-401, Pac-403, Pac-625 and Pac-626 were tested for hemolysis against human red blood cells (RBC). The RBCs with EDTA were rinsed 3 times with PBS (800 g, 10 min) and re-suspended in PBS. The RBCs were diluted into 10% with phosphate-buffered saline and placed 501 μl into each eppendorf. The peptides dissolved in PBS were then added to 50 μl of 10% solution of RBCs and incubated for an hour at 37° C. (final RBC concentration, 5% v/v). The samples were centrifuged at 800 g for 10 min at OD540. Various concentrations of peptides were incubated with pretreated RBC and the percentage of hemolysis determined. The results show that all of the peptides tested were less hemolytic against RBC than other antimicrobial peptides such as melittin (Table 9 and FIG. 5).

TABLE 9
Erythrocyte lysis at various peptide concentrations

| Peptide | % lysis @ | % lysis @ | % lysis @ |
|---|---|---|---|
| name | 5 μg/ml | 50 μg/ml | 500 μg/ml |
| Pac 401 L | 0.85 | 6.8 | 14 |
| Pac 403 L | 0.74 | 7.2 | 15 |
| Pac 625 L | 0.82 | 7.3 | 15 |
| Pac 626 L | 0.81 | 7.2 | 14 |

1. The group of isolated and purified cyclic or linear peptides, each with microbicidal activity, described by one of the following formulae:
Xa₁-Naa-Xa₁-Xa₁-Naa-Xa₂
or
Xa₁-Naa-Xa₂-Xa₁-Naa-Xa₁
wherein:
Xa₁ represents a hydrophilic basic amino acid moiety selected from the group consisting of lysine, arginine, and histidine;
Naa represents an unnatural hydrophobic aromatic amino acid moiety selected from the group consisting of (naphtha-1-yl)alanine (1-Nal), (naphtha-2-yl)alanine (2-Nal), (benzothien-3-yl)alanine (Bal), diphenylalanine (Dip), (4,4′-biphen-yl)alanine (Bip), (anthracen-9-yl)alanine (Ath), and (2,5,7-tri-tert-butyl-indol-3-yl)alanine (Tht); and
Xa₂ represents a hydrophobic amino acid moiety selected from the group consisting of valine, leucine, and isoleucine.

2. An isolated and purified peptide chosen from the group of peptides of claim 1, which is selected from the group consisting of

| | |
|---|---|
| K(2-Nal)RR(2-Nal)I | (SEQ ID NO:1) |
| K(2-Nal)RR(2-Nal)V | (SEQ ID NO:2) |
| K(2-Nal)IK(2-Nal)R | (SEQ ID NO:3) |
| K(2-Nal)RR(2-Nal)VR(2-Nal)I | (SEQ ID NO:4) |
| I(2-Nal)RV(2-Nal)RR(2-Nal)K | (SEQ ID NO:5) |
| K(1-Nal)RR(1-Nal)VR(1-Nal)I | (SEQ ID NO:6) |
| K(Bip)RR(Bip)VR(Bip)I | (SEQ ID NO:7) |

3. The isolated and purified peptides of claim 1, wherein the isolated and purified peptides are of linear topology.

4. The isolated and purified peptides of claim 1, wherein the isolated and purified peptides are of cyclic topology.

5. An isolated and purified peptide of claim 1, wherein the isolated and purified peptide is amidated at the C-terminal amino acid.

6. An isolated and purified peptide of claim 1, wherein the peptide is acetylated at the N-terminal amino acid.

7. An isolated and purified peptide of claim 1, wherein the peptide is esterified at the C-terminal amino acid.

8. An isolated and purified peptide of claim 1 which has at least one amino acid altered to a corresponding D-amino acid.

9. A composition comprising an isolated and purified peptide according to claim 1 in a mixture with a carrier or excipient.

10. A pharmaceutical composition which comprises a peptide of claim 1 and a pharmaceutical carrier.

11. A peptide which is the retro-oriented amino acid sequence of a peptide of claim 1.

12. A method to inactivate an endotoxin of Gram-negative bacteria comprising the step of administering an effective amount of an isolated and purified antimicrobial peptide according to claim 1 to a subject in need thereof.

13. A method to treat a microbial or viral infection in a subject comprising the step of administering an effective amount of an isolated and purified antimicrobial peptide according to claim 1 to the subject.

14. A method to inhibit the growth of a microbe comprising the step of administering an effective amount of an isolated and purified antimicrobial peptide according to claim 1 to a subject in need thereof.

15. A peptide of claim 1 which is of the formula Lys-(2-Nal)-Arg-Arg-(2-Nal)-Val-Arg-(2-Nal)-Ile.

16. A pharmaceutical composition which comprises a peptide of claim 15 and a pharmaceutical carrier.


SEQUENCE LISTING (7 sequences)

SEQ ID NO: 1 - protein, 6 aa, Artificial Sequence
  Chemically synthesized synthetic construct
KXRRXI

SEQ ID NO: 2 - protein, 6 aa, Artificial Sequence
  Chemically synthesized synthetic construct
KXRRXV

SEQ ID NO: 3 - protein, 6 aa, Artificial Sequence
  Chemically synthesized synthetic construct
KXIKXR

SEQ ID NO: 4 - protein, 9 aa, Artificial Sequence
  Chemically synthesized synthetic construct
KXRRXVRXI

SEQ ID NO: 5 - protein, 9 aa, Artificial Sequence
  Chemically synthesized synthetic construct
IXRVXRRXK

SEQ ID NO: 6 - protein, 9 aa, Artificial Sequence
  Chemically synthesized synthetic construct
KXRRXVRXI

SEQ ID NO: 7 - protein, 9 aa, Artificial Sequence
  Chemically synthesized synthetic construct
KXRRXVRXI